(19) United States
(12) Patent Application Publication
(10) Pub. No.: US 2017/0303745 A1
(43) Pub. Date: Oct. 26, 2017

(54) Toothbrush Storage Container

(71) Applicant: Alban Ameti, Lincoln Park, NJ (US)

(72) Inventor: Alban Ameti, Lincoln Park, NJ (US)

(21) Appl. No.: 15/495,941

(22) Filed: Apr. 24, 2017

Related U.S. Application Data
(60) Provisional application No. 62/326,201, filed on Apr. 22, 2016.

(51) Int. Cl. A47K 1/09; A61L 2/18; B65D 85/00; A61L 2/26; A47K 1/00; A61L 2/00

(52) U.S. Cl. CPC A47K 1/09; B65D 85/00; A61L 2/26; A61L 2/18; A61L 2/00; A47K 1/00; A61L 2202/182

(57) ABSTRACT

A toothbrush storage container for holding and storing a toothbrush is provided. The toothbrush has a handle and a brush head secured to the handle. The toothbrush storage container comprises a watertight toothbrush receptacle having a base wall, a rear wall, a front wall, a first side wall, and a second side wall. An amount of liquid sanitizing solution is positioned within the toothbrush receptacle. A lid is provided and secured to the rear wall of the toothbrush receptacle. A locking mechanism releasably secures the lid in a closed, watertight position against upper edges of the rear wall, the front wall, the first side wall, and the second side wall. An ingress port is formed in the lid. Upon insertion of the brush head of the toothbrush through the ingress port and into the liquid sanitizing solution, at least a portion of the handle of the toothbrush extends through the lid.

### CLAIM OF PRIORITY

This patent application claims priority under 35 USC 119 (e) (1) from U.S. Provisional Patent Application Ser. No. 62/326,201 filed Apr. 22, 2016, of common inventorship herewith entitled, “Toothbrush Buddy,” which is incorporated herein by reference as though the same were set forth in its entirety.

### FIELD OF THE INVENTION

The present invention pertains to the field of toothbrush storage containers, and more specifically to the field of toothbrush storage containers providing a toothbrush receptacle constructed for containing a liquid sanitizing solution, such as a mouthwash, in which the brush head is submerged between uses.

### BACKGROUND OF THE INVENTION

Brushing one's teeth is one of the most important parts of healthy hygiene. In addition to freshening one's breath and maintaining teeth, proper brushing kills germs and bacteria that grow inside an individual's mouth. A common chronic childhood disease is tooth decay. Children lose more than five hundred million school hours a year due to dental related illness. Adults suffer from various forms of dental disease and these complications often lead to painful oral surgeries and costly trips to the dentist. Health professionals maintain that through education and a concerted effort, the number of adults and children suffering from dental disease can be significantly reduced. To have a healthy smile, the American Dental Association recommends that teeth should be brushed at least twice daily.

The benefits of having clean teeth and a healthy mouth have never been disputed. Most people have little realization that the toothbrush itself can spread more germs than it eliminates. As most people brush their teeth and store their brushes in the bathroom, they fail to realize that these unsanitary rooms are ideal breeding grounds for germs and disease causing bacteria such as salmonella, E. coli, staphylococci, streptococci and campylobacter. These aforementioned bacteria that cause foodborne health problems are also found in bathrooms and unfortunately on toothbrushes. A rather disturbing thought is the fact that millions of germs fly out of the toilet bowl and into the air every time it is flushed, settling on all surfaces including toothbrushes. In addition to this airborne bacteria, there is bacteria which resides in one's mouth and is left behind on a toothbrush and reintroduced into one's mouth with their next brushing, compounding issues of tooth decay, bad breath and periodontal disease. If a person has a cold or flu, these viruses are passed onto a toothbrush, infecting the person again and potentially making it harder to recover. The family toothbrush holder is a means of cross contamination in passing bacteria from one toothbrush to another. Since bacteria multiply so rapidly, a toothbrush provides the kind of moist environment that actually encourages bacterial growth. A germ free toothbrush is important to both oral health and overall physical health as well.

The prior art has put forth several designs for dental care and accessories. Among these are:

U.S. Pat. No. 8,307,979 to Robert Clifford Yuille describes a sanitary toothbrush storage apparatus which comprises at least one holding compartment so shaped and dimensioned as to conveniently accommodate a toothbrush. The compartment is juxtaposed with one or more other compartments disposed in such spaced relationship as to receive a plurality of toothbrushes. The compartments accommodate an aqueous antiseptic solution of an amount sufficient to immerse at least a substantial portion of the toothbrush head such that when awaiting further use, the toothbrush head is hygienically stored in a manner sought to inhibit environmental contamination. Separate holding compartments are arranged in juxtaposed relationship to house small hygiene implements in hygienic conditions in readiment for further use.

U.S. Pat. No. 5,107,987 to Frank J. Palazzolo and Joel L. Stone describes a toothbrush holder having a main container, a lid, and internal separators. The toothbrush main container contains disinfectant in the form of a mouthwash. The container forms a chamber with the disinfectant acting as an immersion agent for loosening materials from a toothbrush. The bottom of the container has a plurality of protrusion support elements which are spaced apart from one another and support a toothbrush dropped into the disinfectant spaced above the bottom of the main container so as to provide trap space for foreign material carried by said toothbrush.

U.S. Pat. No. 4,915,219 to Anthony Ottimo describes a disinfecting toothbrush container for holding a toothbrush and disinfecting bristles of the toothbrush. The container body has a plurality of container chambers formed by dividing the container body by a plurality of substantially vertical container chamber walls. The container chambers are divided transversely by a substantially transverse flexible rubber partition having a slit in each of the container chambers large enough for insertion of the toothbrush. The transverse flexible partition divides each container chamber into an upper portion containing air and a lower portion containing a disinfecting liquid. The lips of each of the slits are firm enough to prevent dirt and germs from the air from entering the disinfecting liquid and flexible enough to sneeze the disinfecting liquid from the bristles of the toothbrush when the toothbrush is drawn through the slit. A reusable lid which can provide a liquid tight closure is provided.

None of these prior art references describe the present invention.

### SUMMARY OF THE INVENTION

It is an object of the present invention to provide a toothbrush receptacle that is particularly constructed for containing a liquid sanitizing solution, such as a mouthwash, in which the brush head is submerged between uses.

The present invention is a toothbrush storage container for holding and storing a toothbrush. The toothbrush has a handle and a brush head secured to the handle. The toothbrush storage container comprises a watertight toothbrush receptacle having a base wall, a rear wall, a front wall opposite the rear wall, a first side wall between the rear wall and the front wall, and a second side wall opposite the first side wall. An amount of liquid sanitizing solution is positioned within the toothbrush receptacle. A lid is provided having a rear edge, a front edge opposite the rear edge, a first side edge between the rear edge and the front edge, and a second side edge opposite the first side edge. The rear edge of the lid is hingedly secured to the rear wall of the toothbrush receptacle. A locking mechanism releasably secures the lid in a closed, watertight position against upper edges of the rear wall, the front wall, the first side wall, and the second side wall. An ingress port is formed in the lid. Upon insertion of the brush head of the toothbrush through the ingress port and into the liquid sanitizing solution, at least a portion of the handle of the toothbrush extends through the lid.

In addition, the present invention includes a method for holding and storing a toothbrush. The toothbrush has a handle and a brush head secured to the handle. The method comprises providing a watertight toothbrush receptacle having a base wall, a rear wall, a front wall opposite the rear wall, a first side wall between the rear wall and the front wall, and a second side wall opposite the first side wall, filling the toothbrush receptacle with an amount of liquid sanitizing solution, hingedly securing a lid to the rear wall of the toothbrush receptacle, releasably securing the lid in a closed, watertight position against upper edges of the rear wall, the front wall, the first side wall, and the second side wall, forming an ingress port in the lid, inserting the brush head of the toothbrush through the ingress port and into the liquid sanitizing solution, resting the brush head of the toothbrush completely within the liquid sanitizing solution, and extending at least a portion of the handle of the toothbrush through the lid.

The present invention further includes a toothbrush storage container for holding and storing a toothbrush. The toothbrush has a handle and a brush head secured to the handle. The toothbrush storage container comprises a watertight toothbrush receptacle having a base wall, a rear wall, a front wall opposite the rear wall, a first side wall between the rear wall and the front wall, and a second side wall opposite the first side wall. An amount of liquid sanitizing solution is positioned within the toothbrush receptacle. A lid is provided having a rear edge, a front edge opposite the rear edge, a first side edge between the rear edge and the front edge, and a second side edge opposite the first side edge. A spring loaded, hinge is mounted between the rear edge of the lid and the rear wall of the toothbrush receptacle and biasing the front edge of the lid into an open position in a general direction away from the front wall. A locking mechanism releasably secures the lid in a closed position. An ingress port is formed in the lid. A rubber seal is mounted within the ingress port for effectively sealing about the handle of the toothbrush. The lid forms a watertight seal when the lid is in a closed position resting against upper edges of the rear wall, the front wall, the first wall, and the second wall of the toothbrush receptacle. Upon insertion of the brush head of the toothbrush through the ingress port and into the liquid sanitizing solution, the brush head of the toothbrush rests completely within the liquid sanitizing solution and at least a portion of the handle of the toothbrush extends through the lid and the rubber seal inhibiting the liquid sanitizing solution from splashing or otherwise spilling from the ingress port.

### BRIEF DESCRIPTION OF THE DRAWINGS

FIG. 1 is a perspective view illustrating a toothbrush storage container, constructed in accordance with the present invention, showing a toothbrush inserted in a receptacle container with a spring loaded-hinged lid in an open position and filled with mouthwash.

FIG. 2 is a perspective view illustrating the toothbrush storage container, constructed in accordance with the present invention, showing the toothbrush inserted in the receptacle container with the spring-loaded hinged lid in a closed position and a suction cup for releasably securing to a surface.

FIG. 3 is a perspective view of a top portion of the toothbrush storage container, constructed in accordance with the present invention, showing a rubber seal for holding the neck of a toothbrush and a push tab for opening the lid of the receptacle container.

FIG. 4 is a perspective view of an inside area of the toothbrush storage container, constructed in accordance with the present invention, showing bristles of a toothbrush submerged in the sanitizing mouthwash liquid contained in the receptacle container and releasably held in place by a bracket mounted to the floor base of the receptacle container.

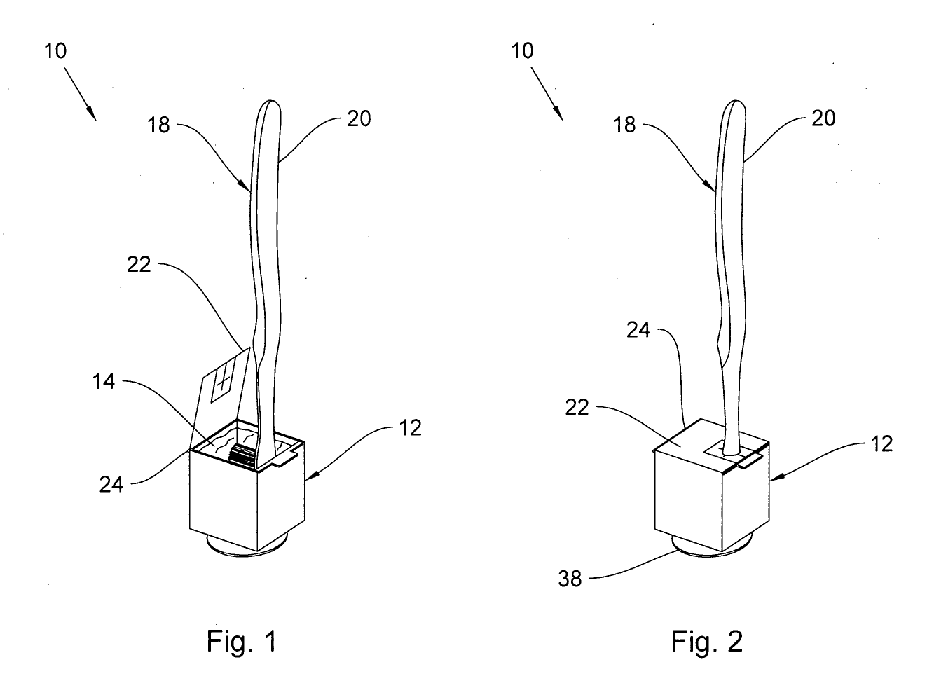

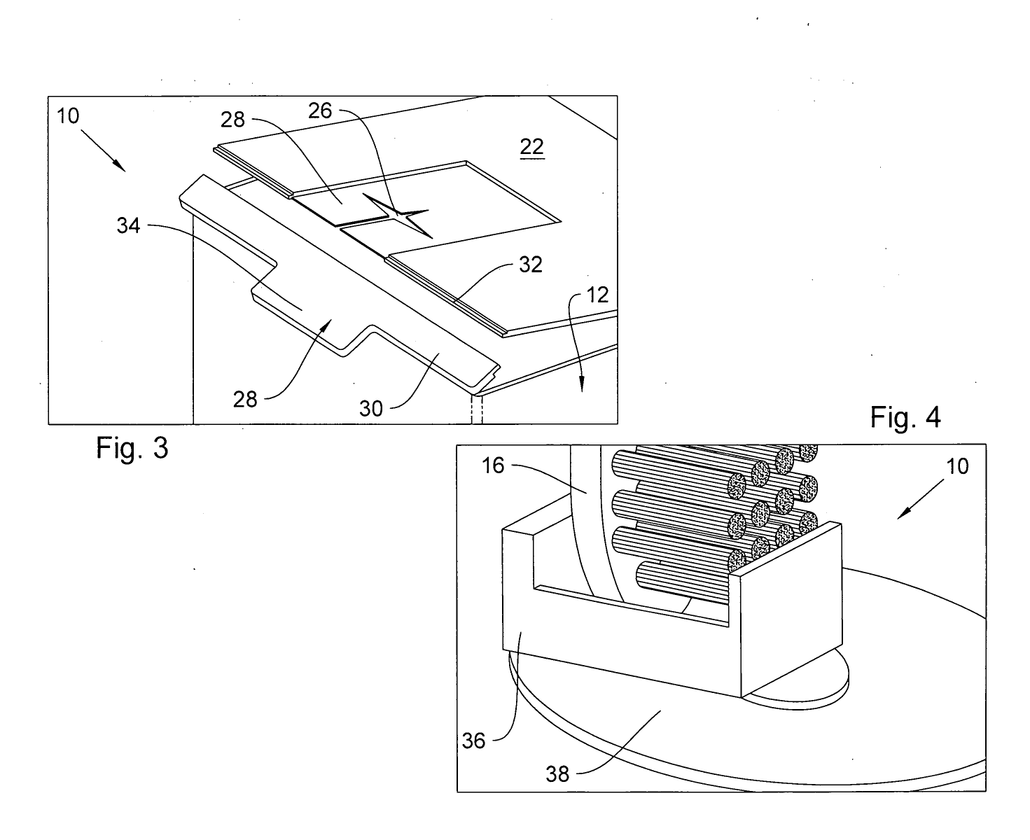

### DETAILED DESCRIPTION OF THE INVENTION

The present invention, hereinafter referred to as a Toothbrush Storage Container, indicated generally at 10, is a watertight toothbrush receptacle 12 constructed for containing a liquid sanitizing solution 14, such as a mouthwash, in which a brush head 16 of a toothbrush 18 is submerged between uses and at least a portion of a handle 20 of the toothbrush 18 extends through a spring-loaded hinged lid 22. The Toothbrush Storage Container 10 inhibits growth of germs and bacteria on the bristles or brush head 16 of the toothbrush 18. The Toothbrush Storage Container 10 provides each household member with a separate cleansing toothbrush receptacle 12 thereby inhibiting the spreading of germs and bacteria between household members.

The toothbrush receptacle 12 of the Toothbrush Storage Container 10 of the present invention has a base wall, a rear wall, a front wall opposite the rear wall, a first side wall between the rear wall and the front wall, and a second side wall opposite the first side wall. Preferably, the Toothbrush Storage Container 10 is constructed from a durable plastic material in a square or cubical in shape and measuring approximately one and one half (1½″) inches to two (2″) inches in height, one approximately (1″) inch to one and a half (1½″) inches in width, and approximately one and one half (1½″) inches to two (2″) inches in depth. The Toothbrush Storage Container 10 are preferably offered in a variety of styles and colors to appeal to individual tastes.

The Toothbrush Storage Container 10 of the present invention includes the lid 22 having a rear edge, a front edge opposite the rear edge, a first side edge between the rear edge and the front edge, and a second side edge opposite the first side edge. The rear edge of the lid 22 is hingedly secured to the rear wall of the toothbrush receptacle 12. Preferably, the lid 22 is hingedly connected to the rear wall of the toothbrush receptacle 12 by a spring loaded, hinge 24 biasing the front edge of the lid 22 into an open position in a general direction away from the front wall of the toothbrush receptacle 12. The lid 22 is preferably a watertight 22 lid such that when the lid 22 is in a closed position resting against upper edges of the rear wall, the front wall, the first wall, and the second wall of the toothbrush receptacle 12, the lid 22 effectively seals the toothbrush receptacle 12 facilitating optimal protection and a spill proof container for maintaining the liquid sanitizing solution 14 therein.

In addition, the Toothbrush Storage Container 10 of the present invention includes a split, soft rubber, ingress port 26 formed in the lid 22 allowing for easy insertion of the brush head 16 of the toothbrush 18 therethrough. The ingress port 26 has a rubber seal 28 for effectively sealing about the toothbrush 18 thereby inhibiting the liquid sanitizing solution 14 from splashing or otherwise spilling from the ingress port. Preferably, the ingress port 26 is centered between the first side edge and the second side edge of the lid 22 nearingly adjacent the front edge of the lid 22 opposite the hinge connection 24.

Additionally, the Toothbrush Storage Container 10 of the present invention includes a locking mechanism 28 consisting of a locking portion 30 interacting with a shoulder 32 formed in the front edge of the lid 22. The locking portion 30 is pivotally secured to the upper edge of the front wall of the toothbrush receptacle 12 and pivots in a generally upward direction over the shoulder 32 of the lid 22 thereby securing the lid 22 in a closed position. Preferably, a push tab 34 extends from the locking portion 30 allows a user to more easily manipulate the locking portion 30 into the desired position. The locking mechanism 28, when the locking portion 30 is moved over the shoulder 32 formed in the lid 22, overcomes the bias of the spring hinge 24 and releasably secures the lid 22 in a watertight, sealed, closed position.

Furthermore, the Toothbrush Storage Container 10 of the present invention includes a bracket 36 mounted to the base wall within the toothbrush receptacle 12. The bracket 36 includes at least a pair of opposite raised side walls for releasably, frictionally holding the brush head 16 of the toothbrush 18 in an upright position.

The Toothbrush Storage Container 10 of the present invention further includes a suction cup 38 mounted to the base wall on the outside of the toothbrush receptacle 12. The suction cup 38 durably, releasably adheres the toothbrush receptacle 12 to a bathroom surface such as a counter or vanity.

Use of the Toothbrush Storage Container 10 of the present invention is very simple and straightforward. A user selects a desired color of toothbrush receptacle 12. A young bachelor may choose a solid blue container. A large family may select a package of whimsically designed receptacles that appeal to the children. Finding an appropriate surface in the bathroom for placing the Toothbrush Storage Container, a user then places the suction cup 38 of the present invention flush on the selected surface. Opening the bottle of the chosen mouthwash brand 14, the user fills the toothbrush receptacle 12 with the liquid 14, closing the lid 22 and releasably locking it shut by means of the locking mechanism 28. The toothbrush 18 is placed into the Toothbrush Storage Container and its stored solution 14 with the toothbrush head 16 of the toothbrush 18, and thus the bristles, resting in the bracket 36 submerged in the liquid 14. When ready to brush their teeth, the user removes the toothbrush 18 from the liquid sanitizer 14 and dispenses toothpaste onto the brush head 16 of the toothbrush 18. Rinsing the brush head clean after use, the user returns the toothbrush 18 to the Toothbrush Storage Container 10 until ready to use again. In between uses, the sanitizing mouthwash solution 14 is working to rid the brush head 16 of the toothbrush 18 of any airborne or mouth sourced germs and bacteria. Mouthwash 14 preferably is discarded and replaced in the toothbrush receptacle 12 after each use.

The Toothbrush Storage Container 10 of the present invention effectively reduces the spread of harmful germs and bacteria that thrive around and on a toothbrush 18. Providing a convenient holder for toothbrushes 18, the Toothbrush Storage Container 10 eliminates any need to place toothbrushes 18 on a bathroom counter or in another container that is potentially exposed to germs. The mouthwash solution 14 immerses the brush head in protective liquid 14 between uses. Users have a fresh toothbrush 18 for each use which greatly reduces prevalence of flu bugs, colds, infections, and gum diseases associated with bathroom germs and bacteria. The mouthwash solution 14 is easily refreshed as needed. Compact and lightweight, the Toothbrush Storage Container 10 is packable into any shaving kit, travel bag or carry on, and is effectively used while traveling on vacation or business. Durably constructed of a high quality materials and providing a safe and sanitary way of keeping toothbrushes free of harmful bacteria, the Toothbrush Storage Container 10 will withstand many years of continued use.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. A toothbrush storage container for holding and storing a toothbrush, the toothbrush having a handle and a brush head secured to the handle, the toothbrush storage container comprising:
a watertight toothbrush receptacle having a base wall, a rear wall, a front wall opposite the rear wall, a first side wall between the rear wall and the front wall, and a second side wall opposite the first side wall;
an amount of liquid sanitizing solution within the toothbrush receptacle;
a lid having a rear edge, a front edge opposite the rear edge, a first side edge between the rear edge and the front edge, and a second side edge opposite the first side edge, the rear edge of the lid hingedly secured to the rear wall of the toothbrush receptacle;
a locking mechanism for releasably securing the lid in a closed, watertight position against upper edges of the rear wall, the front wall, the first side wall, and the second side wall; and
an ingress port formed in the lid;
wherein upon insertion of the brush head of the toothbrush through the ingress port and into the liquid sanitizing solution, the brush head of the toothbrush rests completely within the liquid sanitizing solution and at least a portion of the handle of the toothbrush extends through the lid.

2. The toothbrush storage container of claim 1 wherein the liquid sanitizing solution inhibits growth of germs and bacteria on the brush head of the toothbrush.

3. The toothbrush storage container of claim 1 wherein the toothbrush receptacle is constructed from a durable plastic material in a square or cubical in shape.

4. The toothbrush storage container of claim 1 and further comprising:
a spring loaded, hinge biasing the front edge of the lid into an open position in a general direction away from the front wall.

5. The toothbrush storage container of claim 1 wherein the locking mechanism includes a locking portion interacting with a shoulder formed in the front edge of the lid, the locking portion pivotally secured to the upper edge of the front wall of the toothbrush receptacle and pivotable in a generally upward direction over the shoulder of the lid thereby overcoming the bias of the spring hinge and releasably securing the lid in a closed position.

6. The toothbrush storage container of claim 5 and further comprising:
a push tab extending from the locking portion.

7. The toothbrush storage container of claim 1 wherein the lid forms a watertight seal when the lid is in a closed position resting against upper edges of the rear wall, the front wall, the first wall, and the second wall of the toothbrush receptacle.

8. The toothbrush storage container of claim 1 wherein the ingress port has a rubber seal for effectively sealing about the handle of the toothbrush thereby inhibiting the liquid sanitizing solution from splashing or otherwise spilling from the ingress port.

9. The toothbrush storage container of claim 1 wherein the ingress port is centered between the first side edge and the second side edge of the lid nearingly adjacent the front edge of the lid opposite the hinge connection.

10. The toothbrush storage container of claim 1 and further comprising:
a bracket mounted to the base wall within the toothbrush receptacle.

11. The toothbrush storage container of claim 10 wherein the bracket includes at least a pair of opposite raised side walls for releasably, frictionally holding the brush head of the toothbrush in an upright position.

12. The toothbrush storage container of claim 1 and further comprising:
a suction cup mounted to the base wall on the outside of the toothbrush receptacle.

13. A method for holding and storing a toothbrush, the toothbrush having a handle and a brush head secured to the handle, the method comprising:
providing a watertight toothbrush receptacle having a base wall, a rear wall, a front wall opposite the rear wall, a first side wall between the rear wall and the front wall, and a second side wall opposite the first side wall;
filling the toothbrush receptacle with an amount of liquid sanitizing solution;
hingedly securing a lid to the rear wall of the toothbrush receptacle;
releasably securing the lid in a closed, watertight position against upper edges of the rear wall, the front wall, the first side wall, and the second side wall;
forming an ingress port in the lid;
inserting the brush head of the toothbrush through the ingress port and into the liquid sanitizing solution;
resting the brush head of the toothbrush completely within the liquid sanitizing solution; and
extending at least a portion of the handle of the toothbrush through the lid.

14. A toothbrush storage container for holding and storing a toothbrush, the toothbrush having a handle and a brush head secured to the handle, the toothbrush storage container comprising:
a watertight toothbrush receptacle having a base wall, a rear wall, a front wall opposite the rear wall, a first side wall between the rear wall and the front wall, and a second side wall opposite the first side wall;
an amount of liquid sanitizing solution within the toothbrush receptacle;
a lid having a rear edge, a front edge opposite the rear edge, a first side edge between the rear edge and the front edge, and a second side edge opposite the first side edge;
a spring loaded, hinge mounted between the rear edge of the lid and the rear wall of the toothbrush receptacle and biasing the front edge of the lid into an open position in a general direction away from the front wall;
a locking mechanism for releasably securing the lid in a closed, position;
an ingress port formed in the lid;
a rubber seal mounted within the ingress port for effectively sealing about the handle of the toothbrush;
wherein the lid forms a watertight seal when the lid is in a closed position resting against upper edges of the rear wall, the front wall, the first wall, and the second wall of the toothbrush receptacle; and
wherein upon insertion of the brush head of the toothbrush through the ingress port and into the liquid sanitizing solution, the brush head of the toothbrush resting completely within the liquid sanitizing solution and at least a portion of the handle of the toothbrush extends through the lid and the rubber seal inhibiting the liquid sanitizing solution from splashing or otherwise spilling from the ingress port.

15. The toothbrush storage container of claim 14 wherein the locking mechanism includes a locking portion interacting with a shoulder formed in the front edge of the lid, the locking portion pivotally secured to the upper edge of the front wall of the toothbrush receptacle and pivotable in a generally upward direction over the shoulder of the lid thereby overcoming the bias of the spring hinge and releasably securing the lid in a closed position.

16. The toothbrush storage container of claim 15 and further comprising:
a push tab extending from the locking portion.

17. The toothbrush storage container of claim 14 wherein the ingress port has.

18. The toothbrush storage container of claim 14 wherein the ingress port is centered between the first side edge and the second side edge of the lid nearingly adjacent the front edge of the lid opposite the hinge connection.

19. The toothbrush storage container of claim 14 and further comprising:
a bracket mounted to the base wall within the toothbrush receptacle.
wherein the bracket includes at least a pair of opposite raised side walls for releasably, frictionally holding the brush head of the toothbrush in an upright position.

20. The toothbrush storage container of claim 14 and further comprising:
a suction cup mounted to the base wall on the outside of the toothbrush receptacle.
